(19) United States
(12) Patent Application Publication
(10) Pub. No.: US 2004/0215243 A1
(43) Pub. Date: Oct. 28, 2004

(54) Implantable medical device with piezoelectric transformer

(76) Inventors: Richard P.M. Houben, Lanaken (BE); Mark A. Christopherson, Shoreview, MN (US); Adrianus P. Donders, Andover, MN (US); Robert Leinders, Limbricht (NL); Curtis D. Deno, Andover, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/424,581

(22) Filed: Apr. 25, 2003

(51) Int. Cl. A61N 1/39

(52) U.S. Cl. 607/5

(57) ABSTRACT

In general, the invention is directed to an IMD having a piezoelectric transformer to convert battery power to operating power. The piezoelectric transformer serves to convert voltage levels produced by a battery in the IMD to voltage levels appropriate for IMD operation. In contrast to electromagnetic transformers and charge pump arrays, a piezoelectric transformer offers small size and low profile, as well as operational efficiency. In addition, in an implantable cardiac or neurostimulation device, the piezoelectric transformer provides electrical isolation that avoids circuit-induced cross currents between different electrodes.

### FIELD OF THE INVENTION

The invention relates to implantable medical devices and, more particularly, to power conversion devices for implantable medical devices.

### BACKGROUND OF THE INVENTION

Implantable medical devices (IMDs), such as implantable cardiac pacemakers, pacemaker-cardioverter-defibrillators, neurostimulators, drug pumps, and the like, generally make use of battery power to support the output and functionality of such devices. In many cases, the battery delivers power with a voltage or current level that must be converted upward for use by the IMD.

An IMD such as a defibrillator, for example, requires a voltage level that is often two to five times the voltage level of the battery. For this reason, the defibrillator typically incorporates a charge pump capacitor array that generates charge with the appropriate voltage level and stores the charge on a holding capacitor. The array of pump capacitors and the holding capacitor contribute to the size, cost and complexity of the IMD. In addition, the pump capacitors are typically coupled in series, reducing capacitance and thereby contributing to voltage droop at higher voltages.

Some IMDs, like defibrillators, incorporate electromagnetic transformers to provide power conversion. However, electromagnetic transformers present considerable size disadvantages, increasing the bulk and profile of the IMD. In addition, an electromagnetic transformer can increase charging time between pulses or shocks, and be susceptible to shorts and other malfunctions. As a further drawback, electromagnetic transformers create and are susceptible to electromagnetic interference, e.g., interference caused by magnetic resonance imaging (MRI).

To deliver power from the holding capacitor, defibrillators ordinarily incorporate an output stage with control circuitry and low impedance switches for high voltage generation and pulse delivery. To achieve different voltage levels on a selective basis, complex switch networks are often required. Like a charge pump array or electromagnetic transformer, the output stage circuitry adds to the size, cost and complexity of the device. Other IMDs, such as implantable drug pumps and neurostimulators, have similar power requirements.

### BRIEF SUMMARY OF THE INVENTION

In general, the invention is directed to an IMD having a piezoelectric transformer to convert battery power to operating power. The piezoelectric transformer serves to convert voltage levels produced by a battery in the IMD to voltage levels appropriate for IMD operation. In contrast to electromagnetic transformers and charge pump arrays, a piezoelectric transformer offers small size and low profile, as well as operational efficiency. In addition, in an implantable cardiac or neurostimulation device, the piezoelectric transformer provides electrical isolation that avoids circuit-induced cross currents between different electrodes.

In general, the piezoelectric transformer includes two or more piezoelectric resonators. The piezoelectric resonators are mechanically coupled to one another, but electrically insulated. An input circuit, coupled to a battery, generates an input signal near a resonant frequency of an input resonator. In some embodiments, the input circuit may be a pulse frequency modulation circuit. The input resonator receives the input signal, and generates mechanical vibration due to the piezoelectric converse effect. An output resonator transduces the mechanical vibration to generate an output signal at a second voltage level, due to the piezoelectric direct effect.

The IMD uses the output signal to support device operation. The IMD may be, for example, an implantable cardiac pacemaker, pacemaker-cardioverter-defibrillator, a neurostimulator, a drug pump, or the like. Accordingly, the IMD may use the output signal generated by the piezoelectric transformer to generates pacing pulses, cardioversion shocks, defibrillation shocks, or neurostimulation pulses. Alternatively, the IMD may use the output signal to power components within the IMD. For example, the IMD may use the output signal to power a pump for delivery of drugs or other therapeutic agents.

In one embodiment, the invention provides an implantable medical device comprising a battery to deliver a first voltage, and a piezoelectric transformer to convert the first voltage to a second voltage greater than the first voltage.

In another embodiment, the invention provides an implantable medical device comprising a battery to deliver a first voltage, a piezoelectric transformer to convert the first voltage to a second voltage greater than the first voltage, wherein the piezoelectric transformer includes a first resonator that generates mechanical vibration in response to the input signal, and a second resonator that generates an output signal in response to the mechanical vibration, an input circuit to drive the piezoelectric transformer with the input signal, a hold capacitor, and a charging circuit that applies the output signal to charge the hold capacitor.

In a further embodiment, the invention provides a method comprising converting a first voltage to a second voltage with a piezoelectric transformer, wherein the second voltage greater than the first voltage, and applying the second voltage to charge a hold capacitor within an implantable medical device.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic view illustrating a piezoelectric transformer.

FIG. 2 is a block diagram illustrating an implantable medical device incorporating a piezoelectric transformer.

FIG. 3 is a block diagram illustrating an implantable cardioverter-defibrillator incorporating a piezoelectric transformer.

FIG. 4 is a block diagram illustrating a charging circuit for use in the implantable cardioverter-defibrillator of FIG. 3.

FIG. 5 is a block diagram illustrating an implantable drug pump incorporating a piezoelectric transformer.

FIG. 6 is a block diagram illustrating a pump drive circuit for use in the implantable drug pump of FIG. 5.

FIG. 7 is a circuit diagram illustrating an input circuit to drive a piezoelectric transformer.

FIG. 8 is a schematic view illustrating a charging circuit incorporating a piezoelectric transformer.

### DETAILED DESCRIPTION OF THE INVENTION

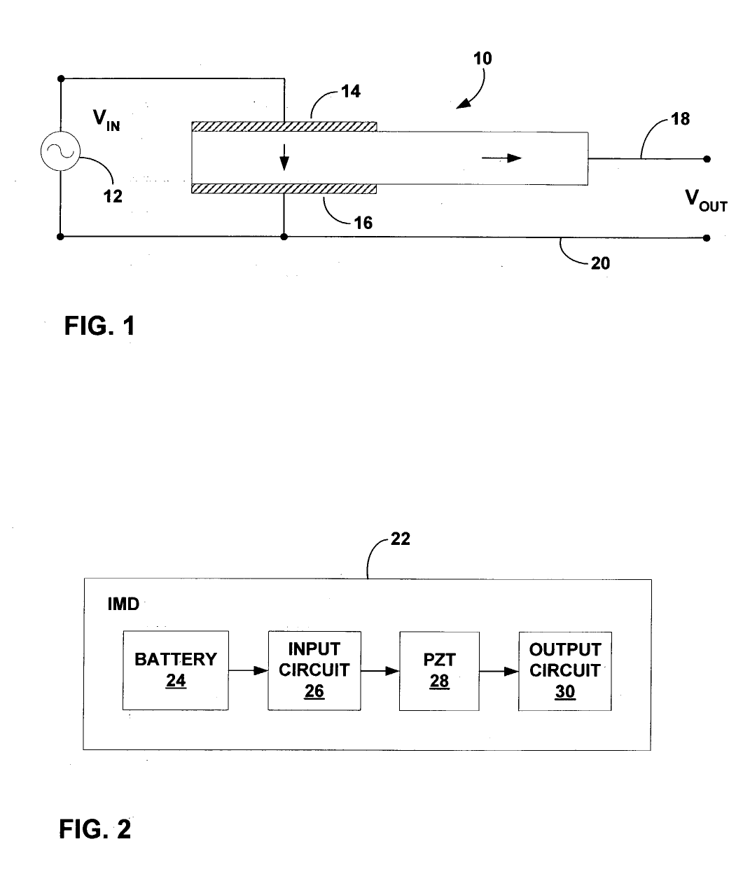

FIG. 1 is a schematic view illustrating a piezoelectric transformer 10. An input circuit 12 drives piezoelectric transformer 10 with an input signal VIN having a frequency matched approximately to the resonant frequency of piezoelectric transformer 10. Piezoelectric transformer 10 includes a first (input) resonator sandwiched between electrodes 14, 16, and a second (output) resonator having an output 18 that generates an output signal VOUT. A ground line 20 serves as reference.

As described herein, piezoelectric transformer 10 serves to convert a first voltage to a second voltage higher than the first voltage within an IMD. The first voltage is generated with power delivered by a battery within the IMD. The second voltage is applied to support operation of the IMD cardioverter-defibrillator. Alternatively, the second voltage may be applied to drive a pump in an implantable drug pump.

In contrast to electromagnetic transformers and charge pump arrays, piezoelectric transformer 10 offers a small size and low profile, as well as operational efficiency. For example, some commercially available piezoelectric transformers are known to offer 80 to 90 percent operational efficiency. In addition, in an implantable cardiac or neurostimulation device, piezoelectric transformer 10 provides electrical isolation that avoids circuit-induced cross currents between different electrodes.

In operation, the first and second resonators of piezoelectric transformer 10 are mechanically coupled to one another, but electrically insulated from one another. Note that the shape, dimensions and form factor of piezoelectric transformer 10 may be very flexible, and subject to wide variation. For example, a piezoelectric transformer 10 having a planar, circular or even toroidal shape is possible, and may be desirable given space constraints within an implantable medical device.

Input circuit 12, coupled to a battery (not shown in FIG. 1), generates the input signal VIN near a resonant frequency of the input resonator. In response, the input resonator generates mechanical vibration, due to the piezoelectric converse effect. The output resonator transduces the mechanical vibration to generate output signal VOUT at a second voltage level, due to the piezoelectric direct effect.

FIG. 2 is a block diagram illustrating an IMD 22 incorporating a piezoelectric transformer (PZT) 28. As shown in FIG. 2, IMD 22 includes a battery 24 that provides power to an input circuit 26. The power delivered by battery 24 has a first voltage level that is generally insufficient to support IMD functions such as delivery of electrical stimulation pulses or shocks, or driving of a pump or other component. Input circuit 26 generates an input signal to drive PZT 28. In response, PZT 28 generates an output signal at a second voltage greater than the first voltage. In some embodiments, if desired, PZT 28 could be selected to produce a second voltage that is less than the first voltage. For delivery of electrical stimulation pulses or shocks, or driving of a pump, however, it will be desirable that the second voltage be significantly greater than the first voltage.

Output circuit 30 applies the second voltage to generate a stimulation pulse or shock, or drive a pump or other component. In the example in which IMD 22 is an implantable cardioverter-defibrillator, the first voltage provided by battery 24 may be less than or equal to approximately 10 volts, whereas the second voltage delivered by PZT may be in excess of 700 volts, and even 800 volts.

Hence, the first voltage delivered by battery 24 may be less than ten percent of the second voltage and, in many, cases less than five percent of the second voltage. The second voltage may be provided directly from PZT 28. Alternatively, in the case of an implantable cardioverter-defibrillator, the second voltage may be generated for output circuit 30. For example, output circuit 30 may include a charging circuit that applies the output signal from PZT 28 to charge a hold capacitor to the second voltage level.

In this manner, IMD 22 uses the output signal of PZT 28 to support device operation. The IMD may be, for example, an implantable cardiac pacemaker, pacemaker-cardioverter-defibrillator, a neurostimulator, a drug pump, or the like. Accordingly, the IMD may use the output signal generated by the piezoelectric transformer to generates pacing pulses, cardioversion shocks, defibrillation shocks, or neurostimulation pulses. Alternatively, the IMD may use the output signal to power components within the IMD. For example, the IMD may use the output signal to power a pump for delivery of drugs or other therapeutic agents.

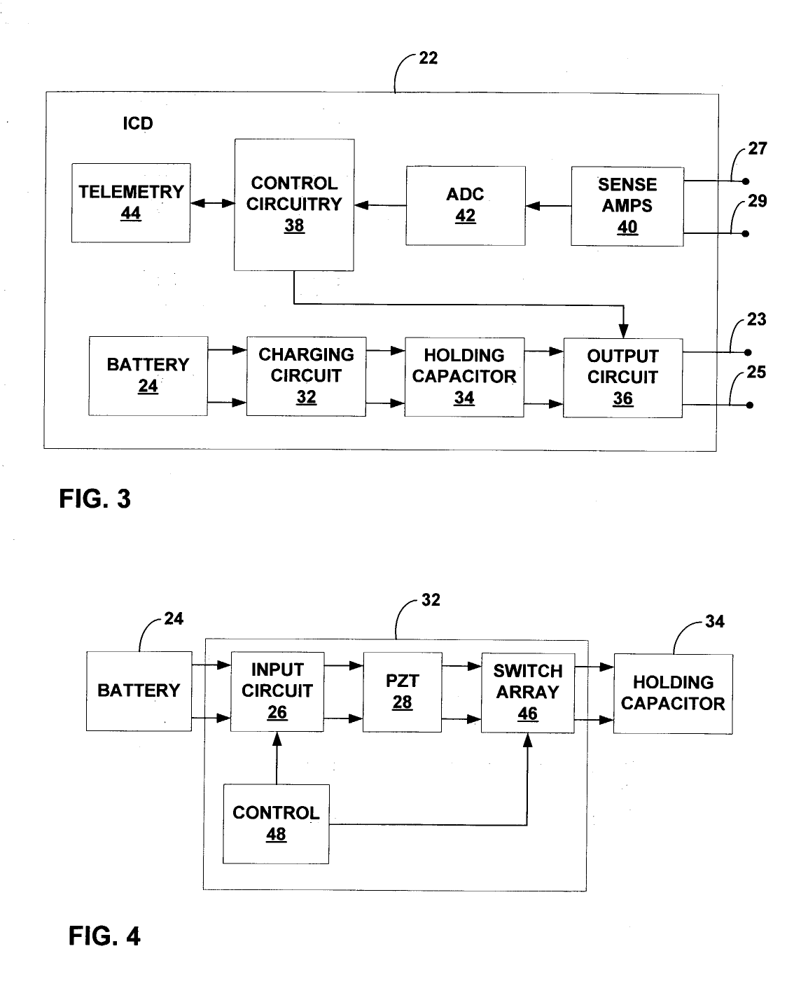

FIG. 3 is a block diagram illustrating an implantable cardioverter-defibrillator (ICD) 22 incorporating a piezoelectric transformer. As shown in FIG. 3, ICD 22 includes battery 24, a charging circuit 32, a holding capacitor 34, and an output circuit 36 coupled to one or more stimulation electrodes 23, 25 deployed within a heart via implantable leads. Control circuit 38 controls output circuit 36 to deliver cardioversion and/or defibrillation shocks via stimulation electrode 23 or stimulation electrode 25. As an example, stimulation electrode 23 may be carried by a right atrial lead and stimulation electrode 25 may be carried by a right ventricular lead.

One or more sense amplifiers 40 receive cardiac signals via sense electrodes 27, 29. Sense electrodes 27, 29 are deployed within the heart via implantable leads. For example, sense electrode 27 may be carried by a right atrial lead and sense electrode 29 may be carried by a right ventricular lead. An analog-to-digital converter (ADC) 42 converts the sensed cardiac signal to digital values for processing and analysis by control circuitry 38, which may include a microprocessor, digital signal processor, ASIC, FPGA, or other equivalent logic circuitry. ICD 22 further includes a telemetry circuit 44 for wireless communication with an external programmer.

As will be described, charging circuit 32 includes a piezoelectric transformer to convert a first voltage provided by battery 24 to a second voltage substantially higher than the first voltage to charge holding capacitor 334. Holding capacitor 34 maintains a store of energy for immediate, on-demand output via output circuit 36 and one or both of stimulation electrodes 23, 25. Charging circuit 32 preferably provides rapid recharging of holding capacitor 34, e.g., on the order of ten seconds or less. A piezoelectric transformer, in accordance with the invention, can be configured and combined with appropriate input and output circuitry to support such rapid recharging of holding capacitor 34.

FIG. 4 is a block diagram illustrating a charging circuit 32 for use in ICD 22 of FIG. 3. As shown in FIG. 4, charging circuit 32 includes an input circuit 26 that uses power provided by battery 24 to generate an input signal for application to PZT 28. A switch array 46 applies the output signal generated by PZT 28 to charge holding capacitor 34. A control circuit 48 controls input circuit 26 and switch array 46 to maintain operation of PZT 28.

In some embodiments, for example, holding capacitor 34 may include two or more capacitors. In this case, control circuit 48 may be configured to control switch array to couple the holding capacitors in series during a charging stage, and couple the holding capacitors in parallel following the charging stage. In this manner, the capacitors form a lower capacitance for charging and then add when coupled in parallel to provide a combined capacitance for delivery of cardioversion or defibrillation shocks.

Charging circuit 32 may be useful in generating higher voltages, e.g., 3-5 times the battery voltage, for the stimulation output stage in bradycardia or neurostimulation therapies. Application of a piezoelectric transformer may permit the number of capacitors to be reduced to a single hold capacitor. In this manner, the piezoelectric transformer can be used to reduce the size, complexity, and number of components in the IMD. An equal hold capacitance for various output amplitudes also helps to ensure equal droops over the pulse duration and therefore equal recharge efficacy.

Advantageously, the piezoelectric transformer also may enable the realization of an IMD that is free of circuit-induced inter-channel cross-current. For example, inclusion of a piezoelectric transformer provides electrical isolation, and thereby circumvents possible current paths from the stimulation electrode to other available electrodes. Subsequent output stage circuitry, i.e., from the piezoelectric output resonator toward the stimulation electrode, should be floating and therefore powered by the transformer. Advantages of reduced inter-channel cross current include more accurate sensing on non-stimulation channels. Also, better control of current vectors in multi-electrode stimulation can be achieved. In general, the use of a piezoelectric transformer permits undesired crosstalk between electrodes to be eliminated via circuit isolation.

In addition, the inclusion of a piezoelectric transformer provides resistance to electromagnetic interference. Specifically, piezoelectric elements are insensitive to electromagnetic interference. Accordingly, the charging cycle performance is unaffected by presence of electromagnetic interference, e.g., from MRI procedures or emissions from equipment within the environment occupied by the patient. In addition, during the charging cycle, the piezoelectric transformer will not generate electromagnetic interference that disrupts telemetry sessions.

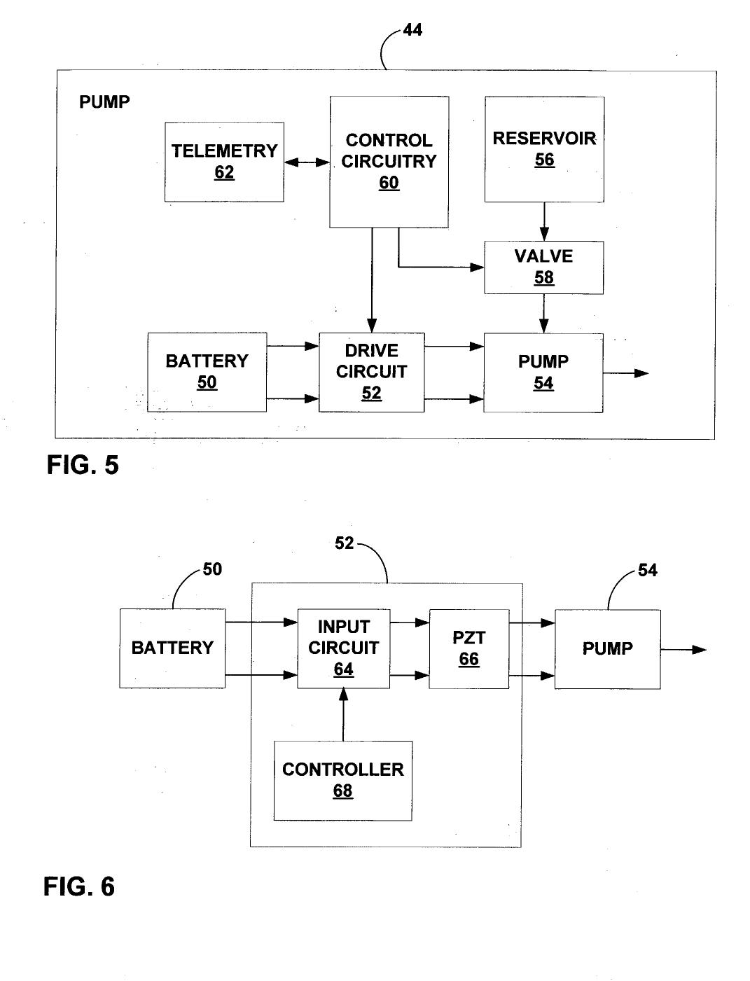

FIG. 5 is a block diagram illustrating an implantable drug pump 44 incorporating a piezoelectric transformer. As shown in FIG. 5, pump 44 includes a battery 50 coupled to a drive circuit 52. Drive circuit 52 drives a pump 54 for delivery of a drug or other fluid substance to a patient via an implanted catheter. In accordance with the invention, drive circuit 52 includes a piezoelectric transformer to convert voltage levels produced by battery 50 to a voltage level suitable to drive pump 54.

For example, pump 54 may comprise a piezoelectric pump that requires high drive voltages. Piezoelectric pump motors typically require a high voltage to actuate the piezo elements of the motor. The voltages to drive the piezoelectric pump may range from 25 volts to 150 volts, and typically draw very little current.

Alternatively, pump 54 may be an electro-osmotic-flow pump that uses a high voltage to generate an electric field across a capillary tube to cause fluid movement. In particular, the electrical field results in the flow of ions through the tube. A buffered ion solution is combined with the drug, resulting in a controlled drug flow rate.

Implantable drug pump 44 further includes a drug reservoir 56, a control valve 48 to release the drug from reservoir 56 for delivery by pump 54. Control circuitry 60 controls valve 58 and drive circuitry to thereby control delivery of the drug. Pump 44 further includes a telemetry circuit 62 to permit wireless communication with an external programmer.

FIG. 6 is a block diagram illustrating a pump drive circuit 52 for use in the implantable drug pump 44 of FIG. 5. As shown in FIG. 6, pump drive circuit 52 includes an input circuit 64 that receives power from battery 50 and generates an input signal at approximately a resonant frequency of an input resonator in PZT 66. PZT 66 generates an output signal in response to the input signal applied by input circuit 64. The output signal has a voltage level that is higher than the voltage level of battery 50. The output signal of PZT 66 is applied to pump 54, directly or via signal conditioning circuitry, to drive the pump motor. A controller 68 may be provided to control operation of input circuit 64.

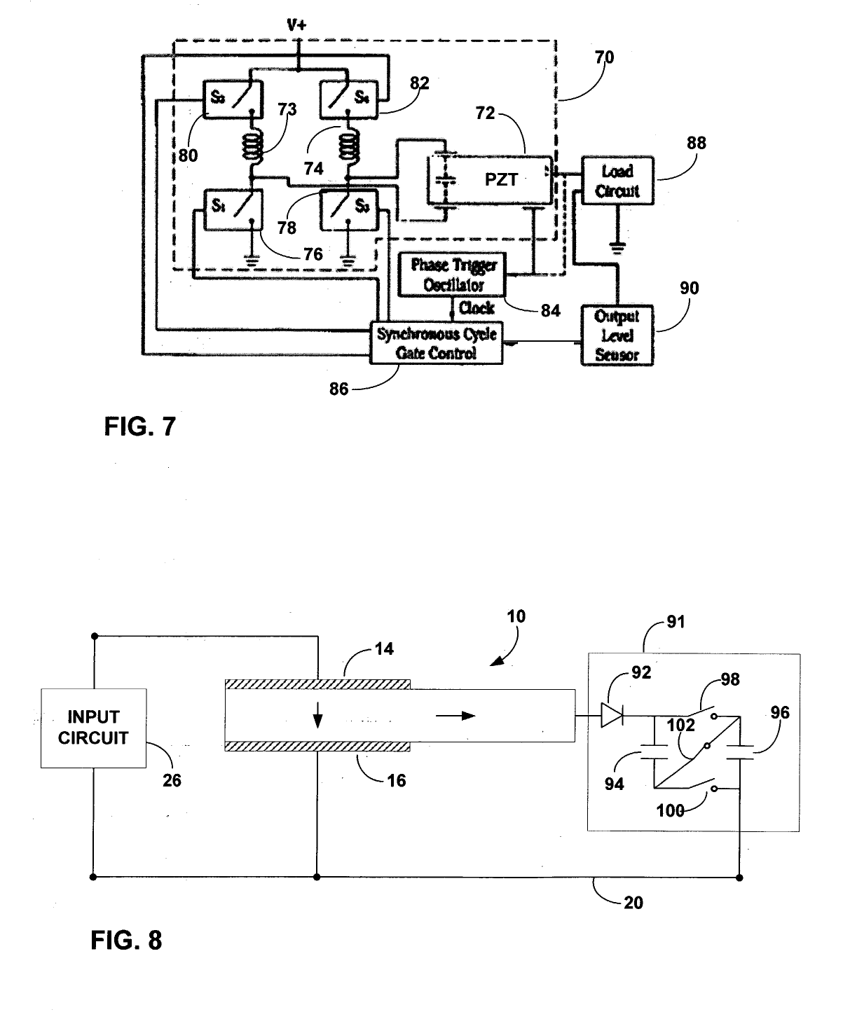

FIG. 7 is a circuit diagram illustrating an input circuit 70 to drive a piezoelectric transformer in the embodiments of any of FIGS. 1-6. In the example of FIG. 7, input circuit 70 comprises a pulse frequency modulation drive circuit. Input circuit 70 drives PZT 72, which has a resonant frequency and input and output resonators. A frequency feedback network is connected to PZT 72. In addition, an output level sensor 90 is coupled between a load circuit 88 and PZT 72. The output of output level sensor 90 is coupled to synchronous cycle gate control circuit 86. Synchronous cycle gate control circuit 86 also receives a clock signal from a phase trigger oscillator 84.

Input circuit 70 further includes a first switch (S1) 76 and a third switch (S3) 78 having a capacitance therebetween. A second switch (S4) 80 and a fourth switch (S4) 82 connects a supply voltage V+ to a pair of inductors 73, 74. The supply voltage V+ is derived from a battery provided in the applicable IMD. Second and fourth switch 80, 82 are driven 180 degrees out of phase at the resonant frequency of PZT 72. In this manner, input circuit 70 drives PZT 72 to generate an output signal with a voltage suitable for application to load circuit 88. Load circuit 88 may be, for example, a pump drive circuit for an implantable pump or a charging circuit for an ICD.

FIG. 8 is a schematic view illustrating a charging circuit incorporating a piezoelectric transfomer 10. The charging circuit of FIG. 8 may be incorporated in an ICD as shown in FIGS. 3 and 4 to charge a hold capacitor. As shown in FIG. 8, an input circuit 26 drives PZT 10 to generate an output signal that is applied to a hold capacitor section 91 via diode 92. In the example of FIG. 8, hold capacitor section 91 includes a pair of capacitors 94, 96 and a switch network provided by switches 98, 100, 102.

Switch 102, when closed, couples capacitors 94, 96 in series. Switches 98, 100 coupled capacitors 94, 96 in parallel. Switch 102 is closed during a charging stage so that the output of PZT 10 charges capacitors 94, 96 in series. Upon completion of the charging stage, switches 98, 100 are closed, and switch 102 is opened, to place capacitors 94, 96 in parallel, and thereby combine the capacitances of the capacitors for delivery of charge.

The charging circuit of FIG. 8 promotes rapid charging by enabling charging to start with a smaller capacitance as a result of capacitors 94, 96 being coupled in series by switch 102. As an illustration, using two capacitors 94, 96, 240 microfarads each, connected in series, results in a load impedance of 120 microfarads for PZT 10. With this capacitance, it is much easier to reach a high voltage level in a much shorter time frame. However, switches 98, 100 are then closed to combine the capacitances, and produce a total capacitance of 480 microfarads.

As an example, PZT 10 may charge load capacitor section 91 to a voltage level on the order of 400 to 800 volts for delivery of defibrillation shocks in the 30 to 40 J range within 5 to 10 seconds. Piezoelectric transformer 10 may be a commercially available piezoelectric transformer. As an example, one suitable piezoelectric transformer is the KPN6000 family of piezoelectric transformers, available from CTS Wireless Components of Albuquerque, N. Mex. In some embodiments, two PZTs may be used in parallel to charge different hold capacitor sections that are then combined to form a hold capacitor for discharge.

Many embodiments of the invention have been described. Various modifications can be made without departing from the scope of the claims. For example, a piezoelectric transformer as described herein may be used to power other types of components. As an illustration, a piezoelectric transformer could be used to provide power for programming of Flash memory within an implantable medical device, which sometimes requires higher voltages. These and other embodiments are within the scope of the following claims.

What is claimed is:

1. An implantable medical device comprising:
a battery to deliver a first voltage; and
a piezoelectric transformer to convert the first voltage to a second voltage greater than the first voltage.

2. The device of claim 1, further comprising an input circuit to drive the piezoelectric transformer with an input signal.

3. The device of claim 2, wherein the piezoelectric transformer includes a first resonator that generates mechanical vibration in response to the input signal, and a second resonator that generates an output signal in response to the mechanical vibration.

4. The device of claim 2, wherein the input circuit comprises a pulse frequency modulation circuit, and the input signal is a pulse frequency modulated signal having a frequency approximately equal to a resonant frequency of the first resonator.

5. The device of claim 1, further comprising a hold capacitor and a charging circuit that applies the output signal to charge the hold capacitor.

6. The device of claim 5, wherein the hold capacitor includes a first hold capacitor and a second hold capacitor.

7. The device of claim 6, further comprising a switch array to couple the first and second hold capacitors in series during a charging stage, and to couple the first and second hold capacitors in parallel following the charging stage.

8. The device of claim 1, wherein the device comprises an implantable cardioverter-defibrillator, and the piezoelectric transformer charges a capacitor for delivery of energy to a patient.

9. The device of claim 1, wherein the device comprises an implantable drug pump, and the piezoelectric transformer delivers power to a pump drive circuit.

10. The device of claim 9, further comprising a piezoelectric pump coupled to the pump drive circuit.

11. The device of claim 1, wherein the device comprises an neurostimulator.

12. The device of claim 1, wherein the first voltage is less than ten percent of the second voltage.

13. The device of claim 1, wherein the second voltage is greater than or equal to 500 volts.

14. The device of claim 1, wherein the second voltage is greater than or equal to 800 volts.

15. An implantable medical device comprising:
a battery to deliver a first voltage;
an input circuit to generate an input signal derived from the first voltage;
a piezoelectric transformer to convert the first voltage to a second voltage greater than the first voltage, wherein the piezoelectric transformer includes a first resonator that generates mechanical vibration in response to the input signal, and a second resonator that generates an output signal in response to the mechanical vibration;
a hold capacitor; and
a charging circuit that applies the output signal to charge the hold capacitor

16. The device of claim 15, wherein the input circuit comprises a pulse frequency modulation circuit, and the input signal is a pulse frequency modulated signal having a frequency approximately equal to a resonant frequency of the first resonator.

17. The device of claim 16, wherein the hold capacitor includes a first hold capacitor and a second hold capacitor.

18. The device of claim 17, further comprising a switch array to couple the first and second hold capacitors in series during a charging stage, and to couple the first and second hold capacitors in parallel following the charging stage.

19. The device of claim 15, wherein the device comprises an implantable cardioverter-defibrillator, and the piezoelectric transformer charges a capacitor for delivery of energy to a patient.

20. The device of claim 15, wherein the device comprises an neurostimulator.

21. The device of claim 15, wherein the first voltage is less than ten percent of the second voltage.

22. The device of claim 15, wherein the second voltage is greater than or equal to 500 volts.

23. The device of claim 15, wherein the second voltage is greater than or equal to 800 volts.

24. A method comprising:
converting a first voltage to a second voltage with a piezoelectric transformer, wherein the second voltage is greater than the first voltage; and
applying the second voltage to charge a hold capacitor within an implantable medical device.

25. The method of claim 24, wherein converting the first voltage to a second voltage includes driving a first resonator of the piezoelectric transformer with a first signal to generate mechanical vibration, and transducing the mechanical vibration with a second resonator of the piezoelectric transformer to produce a second signal with the second voltage.

26. The method of claim 24, wherein the hold capacitor includes a first hold capacitor and a second hold capacitor.

27. The method of claim 26, further comprising a switch array to couple the first and second hold capacitors in series during a charging stage, and to couple the first and second hold capacitors in parallel following the charging stage.

28. The method of claim 24, wherein the device comprises an implantable cardioverter-defibrillator, and the piezoelectric transformer charges the hold capacitor for delivery of energy to a patient.

29. The method of claim 24, wherein the device comprises an neurostimulator.

30. The method of claim 24, wherein the first voltage is less than ten percent of the second voltage.

31. The method of claim 24, wherein the second voltage is greater than or equal to 500 volts.

32. The method of claim 24, wherein the second voltage is greater than or equal to 800 volts.
